(19) United States
(12) Patent Application Publication
(10) Pub. No.: US 2006/0074390 A1
(43) Pub. Date: Apr. 6, 2006

(54) Absorbent article dispensing system

(75) Inventors: Cindy Lou Price, Appleton, WI (US); Diane Michele Underhill, Neenah, WI (US); Teresa Marie Zander, Bonduel, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/959,526

(22) Filed: Oct. 6, 2004

(51) Int. Cl. A61B 19/00

(52) U.S. Cl. 604/357

(57) ABSTRACT

An article dispensing system (80) comprises a package (82) and a plurality of personal care articles (20) contained in the package. Each article (20) can include a topsheet (26) and a backsheet (28) operatively connected with the topsheet. Each article (20) also includes a releasable, sequence-attachment mechanism (34) operatively connected to that article. In a particular aspect, the sequence attachment mechanism (34) can be operatively connected to at least a portion of an outward-facing surface of the article. In another aspect, the articles (20) can be connected to provide a sequencing of the articles in an operative zig-zag arrangement. In a further aspect, the package (82) can include an exit mechanism (84) which provides a package opening (86) through which the articles (20) can be removed from the package (82). In a particular configuration, individual articles (20) can be sequentially removed from the package (82), and an individual article can be readily separated from other articles, particularly other following articles.

### FIELD OF THE INVENTION

The present invention relates to a system for dispensing selected articles. More particularly, the present invention can provide a system for dispensing personal care articles, and the articles may be absorbent articles, such as feminine care pads or adult incontinence products.

### BACKGROUND OF THE INVENTION

Personal care articles, particularly personal care absorbent articles are well known in the art. The absorbent articles have been intended to absorb discharged body fluids, such as urine and/or menses. Such articles and products generally comprise a liquid permeable topsheet and a backsheet. Additionally, a fibrous mass or other absorbent body, which can absorb and hold the body fluids, is assembled between the topsheet and backsheet. Incontinence products have typically been employed to absorb liquids, such as urine. Feminine care articles have typically been employed to absorb urine, menses and other vaginal discharges. In particular arrangements, the feminine care articles have included a conventional garment-fastener for securing an individual article in a wearer's undergarment. In further arrangements, the articles have included wing portions which can help to hold the article in place at a selected location in a wearer's undergarment. In some arrangements, the wing portions have been integrally formed with one or more of the preexisting component layers that were employed to construct the article. In other arrangements, the wing portions have been separately provided components that are assembled and affixed to the final product. Conventional wing-fasteners have been employed to secure the wing portions in a desired configuration during ordinary use. The various garment-fasteners and wing-fasteners have included conventional fastening mechanisms, such as adhesive fasteners and mechanical fasteners. The mechanical fasteners have typically included conventional, hook-and-loop fasteners.

The personal care articles have also been contained in various conventional packaging systems. Individual articles have included wrappers, such as individual containment pouches composed of polymer films and/or nonwoven fabrics. Predetermined quantities of articles have also been grouped and contained in conventional packages, such as bags and/or cartons, and the selected packages could be opened to allow a desired extraction of the individual articles.

Conventional packaging systems, however, have not provided desired combinations of convenience and easy access to the individual articles within a package. The packaging systems have been overly cumbersome to use, particularly when the package is partly full and the packaging material is relatively inexpensive. For example, it has been difficult to provide a low cost packaging system that is easy to use with only one hand, and provides a desired ease of access even when the package is partially empty. Additionally, the conventionally employed arrays of packaged articles have been excessively difficult to assemble and manufacture. As a result, there has been a continued need for an improved packaging and dispensing system that can provide a more convenient access to the packaged articles, and can be more efficiently manufactured.

### BRIEF DESCRIPTION OF THE INVENTION

Generally stated, the present invention can provide an article dispensing system 80 which includes a package and a plurality of personal care articles contained in the package. Each article can include a topsheet and a backsheet operatively connected with the topsheet. Each article can also include a releasable, sequence-attachment mechanism operatively connected to that article. In a particular aspect, the sequence-attachment mechanism can be operatively connected to at least a portion of an outward-facing surface of the article. In another aspect, the articles can be connected to provide a sequencing of the articles in an operative zig-zag arrangement. In a further aspect, the package can include an exit mechanism which provides a package opening through which the articles can be removed from the package. Still other aspects can include configurations in which individual articles can be sequentially removed from the package, and in which an individual article can be readily separated from other articles, particularly other subsequent articles.

By incorporating its various aspects and features, the dispensing system of the invention can more efficiently and more conveniently present individual articles to the user. For example, incorporating an interconnected series of articles, with or without wrappers, can help present the next unused article for convenient and easy access and grasping. Additionally, the configuration of the dispensing system can help improve the ease of manufacturing.

### BRIEF DESCRIPTION OF THE DRAWINGS

The various features, aspects and advantages of the present invention will become better understood with reference to the following description, appended claims and accompanying drawings where:

FIG. 1 shows a schematic, partially cut-away view of a representative system for dispensing articles from a package.

FIG. 2 shows a schematic view of a representative package having a panel member which provides an exit slot.

FIG. 3 shows a perspective view of a representative system for dispensing articles from a package.

FIG. 4 shows a partially cut away, top plan view of a bodyside of a representative personal care article.

FIG. 5 shows a view of a representative, longitudinal cross-section through a personal care article of the invention.

FIG. 6 shows a partially cut away, plan view of a bodyside of a representative personal care article having a pair of wing-panels, where the wing-panels are arranged in a laterally-extended position.

FIG. 7 shows a side view of a representative series of individual, wrapped articles connected and sequenced in a zig-zag arrangement.

FIG. 8 shows a side view of a representative series of individual articles connected and sequenced in a zig-zag arrangement, wherein the individual articles have been folded along a transverse fold line.

FIG. 9 shows a partially cut-away, schematic bottom view of a series of individual articles representatively connected in sequence prior to their arrangement in a zig-zag configuration.

FIG. 10 shows a schematic view of representative series of individual articles connected in sequence and arranged into a zig-zag configuration.

FIG. 11 shows a partially cut-away, schematic bottom view of another series of individual articles representatively connected in sequence with a separately provided strip of material prior to their arrangement into a zig-zag configuration.

FIG. 12 shows a schematic, side view of a representative series of individual articles connected in sequence with a separately provided strip and arranged into a zig-zag configuration.

FIG. 13 shows a representative article which includes a wrapper, where a portion of the wrapper is in an opened arrangement.

FIG. 14 shows a schematic view of a representative cross-section of a folded article within a wrapper.

FIG. 15 shows a schematic top view of a representative series of individual articles in a connected sequence of closed wrappers.

FIG. 16 shows a schematic side view of a representative series of individually wrapped articles connected in sequence, as arranged in a zig-zag configuration to provide a plurality of article stacks within a package.

FIG. 16A shows a schematic side view of a representative series of individual wrapped articles connected in sequence, as arranged in another zig-zag configuration to provide a plurality of article stacks within a package.

FIG. 16B a schematic side view of a representative series of individual articles connected in sequence, as arranged in still another zig-zag configuration to provide an array of multiple article stacks when the articles are positioned in a package.

FIG. 17 is a schematic, perspective view of a representative dispensing system having a position advancement mechanism which includes a traction mechanism.

FIG. 18 is a schematic, perspective view of another representative dispensing system having a position advancement mechanism which includes a forcing mechanism.

FIG. 19 is a schematic, perspective view of a dispensing system which includes a representative set of readily collapsible walls.

### DETAILED DESCRIPTION OF THE INVENTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

By the terms “particle,” “particles,” “particulate,” “particulates” and the like, it is meant that the adsorbent material is generally in the form of discrete units. The units can comprise granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

As used herein, the term “nonwoven” refers to a fabric web that has a structure of individual fibers or filaments which are interlaid, but not in an identifiable repeating manner.

As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

“Coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein, the phrase “complex liquid” describes a liquid generally characterized as being a viscoelastic liquid comprising multiple components having inhomogeneous physical and/or chemical properties. It is the inhomogeneous properties of the multiple components that challenge the efficacy of an adsorbent material in the handling of complex liquids. In contrast with complex liquids, simple liquids, such as, for example, urine, physiological saline, water and the like, are generally characterized as being relatively low-viscosity and comprising one or more components having homogeneous physical and/or chemical properties. As a result of having homogeneous properties, the one or more components of simple liquids behave substantially similarly during absorption or adsorption.

Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells.

As used herein, the phrase “absorbent article” refers to devices which absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, tampons, interlabial devices and the like), infant diapers, children's training pants, adult incontinence products and the like; as well as absorbent wipes and covering mats.

Disposable absorbent articles such as, for example, many of the feminine care absorbent products, can include a liquid pervious topsheet, a substantially liquid impervious backsheet operatively connected to the topsheet, and an absorbent core positioned and held between the topsheet and the backsheet. The topsheet is operatively permeable to the liquids that are intended to be held or stored by the absorbent article, and the backsheet may be substantially impermeable or otherwise operatively impermeable to the intended liquids. The absorbent article may also include other components, such as liquid wicking layers, liquid intake layers, liquid distribution layers, transfer layers, barrier layers, and the like, as well as combinations thereof. Disposable absorbent articles and the components thereof, can operate to provide a body-facing surface and a garment-facing surface. As used herein, “body-facing surface” means that surface of the article or component which is intended to be disposed toward or placed adjacent to the body of the wearer during ordinary use, while the “outward surface” or “outward-facing surface” is on the opposite side, and is intended to be disposed to face away from the wearer's body during ordinary use. The outward surface may be arranged to face toward or placed adjacent to the wearer's undergarments when the absorbent article is worn.

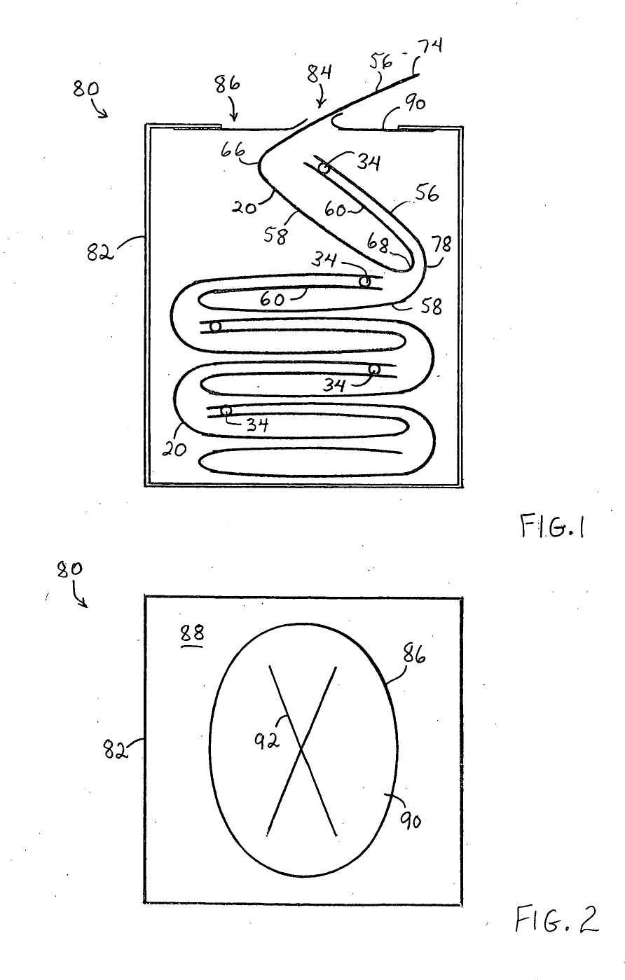

With reference to FIGS. 1-3 and 17-19, the present invention provides a distinctive article dispensing system 80 which includes a package 82 and a plurality of personal care articles 20 contained in the package. Each article 20 can include a topsheet 26, and a backsheet 28 operatively joined or otherwise operatively connected with the topsheet. Each article 20 can also include a releasable, sequence-attachment mechanism 34 operatively joined or otherwise operatively connected to that article. In a particular aspect, the sequence-attachment mechanism 34 can be operatively connected to at least a portion of an outward-facing surface of the article. In another aspect, the articles 20 can be connected to provide a sequencing of the articles in an operative zig-zag arrangement. In a further aspect, the package 82 can include an exit mechanism 84 which provides a package opening 86 through which the articles 20 can be removed from the package 82. In still other aspects, individual articles 20 can be sequentially removed from the package 82, and an individual article can be selectively separated from other articles, particularly other subsequent articles that remain in the package and are configured to follow the last removed article.

By incorporating its various aspects, features and configurations (alone or in desired combinations), the dispensing system of the invention can more efficiently and more conveniently present individual articles to the user. The dispensing system can save time during changing and can be easier for user/caregiver to use. For example, incorporating an interconnected series of articles, with or without wrappers, can help present the next unused article for convenient and easy access and grasping. The interconnected configuration of the articles can help in the dispensing of the articles by pulling the following, unused article up to the top of the dispenser and more conveniently presenting the following article for a subsequent, next-time use. Additionally, the dispensing system can help provide desired manufacturing efficiencies. The interconnected configuration of the articles or their wrappers can improve the ease of manufacturing. The articles or wrappers that are temporarily attached to one another can help entrain and move the articles through the production processes and help to align and regularly sequence the articles during manufacture.

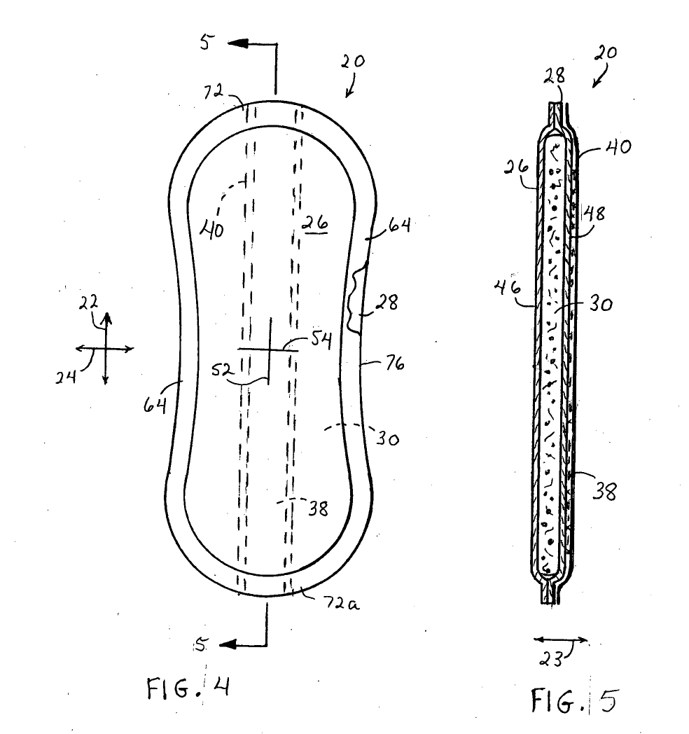

FIGS. 4 through 6 show representative examples of a suitable article 20, such as the shown personal care article, which is configured to incorporate the present invention. The personal care article can be a feminine care article, which can, for example, be a feminine care pad or napkin. The article has an appointed bodyside surface, an appointed garment-side surface, a lengthwise longitudinal-direction 22, a lateral cross-direction 24, a pair of longitudinally-extending, laterally-opposed side edge regions 64, and an intermediate portion 76 which is interposed between a pair of longitudinally opposed end portions 72 (72, 72a). In desired arrangements, each article end portion 72 can be configured to extend over approximately ⅓ of an overall, longitudinal length of the article. Additionally, the intermediate portion 76 can be configured to extend over approximately ⅓ of the overall article length.

Each sequence-attachment mechanism 34 can be configured to releasably connect an end portion of a primary-article 74 to an end portion of a serially-adjacent, sequential article 78. The article also has a longitudinally extending centerline 52 and a laterally extending centerline 54. As representatively shown, the longitudinal dimension of the article is relatively larger than the lateral dimension of the article. In particular configurations, each article 20 can include a baffle or backsheet 28, and a liquid-permeable cover or topsheet 26 which is operatively connected in a facing relation with the backsheet. In particular configurations, each article may further include an absorbent body structure 30 which is operatively positioned and sandwiched between its corresponding backsheet 28 and topsheet 26. As representatively shown, peripheries of the topsheet and backsheet may be substantially entirely coterminous. Alternatively, the peripheries of the topsheet 26 and the backsheet 28 may be partially or entirely non-coterminous.

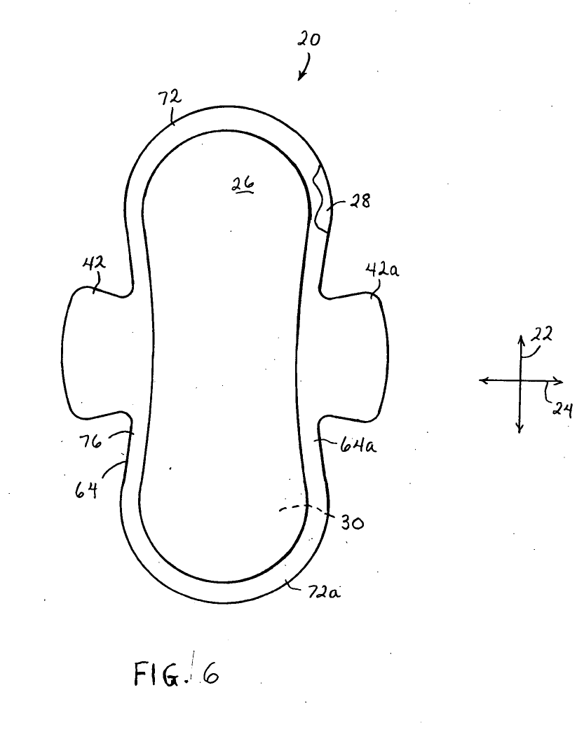

With reference to FIGS. 4 and 6, each article 20, when in its substantially unfolded condition, can have a terminal, perimeter edge, and the perimeter edge can be non-rectangular. In particular arrangements, each article 20, when in its unfolded condition, can have a configuration in which a significant portion of the perimeter edge is curvilinear.

In a desired feature, the article can also include at least one pair of wing-panels 42. A first wing-panel 42 can be attached to a first side edge region 64 in the intermediate portion 76 of the article 20, and a second wing-panel 42a can be attached to a second side edge region 64a in the intermediate portion 76 of the article 20. Each wing-panel 42 (42, 42a) can be configured to wrap about an undergarment of a wearer. A panel fastener 44 can be operatively connected to each wing-panel 42, and each panel fastener 44 can be configured to operatively secure its corresponding wing-panel 42 about the undergarment of the wearer during ordinary use. Each wing-panel 42 can include a panel base section and a panel free-end region, and each panel base section can be operatively connected to its corresponding side edge region 64 of the article 20. Each panel free-end region can extend from its corresponding panel base section, and can be configured to operatively wrap around an appointed region of the wearer's undergarment. For example, the free-end regions of the wing-panels can be wrapped about a crotch region of the wearer's undergarment.

The cover or topsheet 26 may include any material that can be configured to provide the topsheet with an operative level of liquid-permeability. The topsheet may be constructed with one or more layers of suitable materials, and may be a composite material. For example, the topsheet layer can include a woven fabric, a nonwoven fabric, a polymer film, a film-fabric laminate or the like, as well as combinations thereof. Examples of a nonwoven fabric include spunbond fabric, meltblown fabric, coform fabric, a carded web, a bonded-carded-web, a bicomponent spunbond fabric or the like as well as combinations thereof. For example, the topsheet layer can include a woven fabric, a nonwoven fabric, a polymeric film that has been configured to be operatively liquid-permeable, or the like, as well as combinations thereof. Other examples of suitable materials for constructing the topsheet layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable topsheet layer material can include a bonded-carded-web composed of polypropylene and polyethylene, such as has been used as a topsheet stock for KOTEX brand pantiliners. Suitable topsheet materials have been obtainable from Vliesstoffwerk Christian Heinrich Sandler GmbH & Co. KG, a business having an address at Posffach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. In a desired arrangement, the topsheet layer 26 can be configured to be operatively liquid-permeable with regard to the liquids that the article is intended to absorb or otherwise handle. The operative liquid-permeability may, for example be provided by a plurality of pores, perforations, apertures or other openings, as well as combinations thereof, that are present or formed in the topsheet layer. The apertures or other openings can help increase the rate at which bodily liquids can move through the thickness of the topsheet layer and penetrate into the other components of the article (e.g. into the absorbent structure 30). The selected arrangement of liquid-permeability is desirably present at least on an operative portion of the topsheet layer that is appointed for placement on the body-side of the article. The topsheet layer 26 can provide comfort and conformability, and can function to direct bodily exudates away from the body and toward the absorbent structure 30. In a desired feature, the topsheet layer 26 can be configured to retain little or no liquid in its structure, and can be configured to provide a relatively comfortable and non-irritating surface next to the body-tissues of a female wearer. The topsheet layer 26 can be constructed of any material which is also easily penetrated by bodily fluids that contact the surface of the topsheet layer.

The topsheet 26 can also have at least a portion of its bodyside surface treated with a surfactant to render the topsheet more hydrophilic. The surfactant can permit arriving bodily liquids to more readily penetrate the topsheet layer. The surfactant may also diminish the likelihood that the arriving bodily fluids, such as menstrual fluid, will flow off the topsheet layer rather than penetrate through the topsheet layer into other components of the article (e.g. into the absorbent body structure). In a particular configuration, the surfactant can be substantially evenly distributed across at least a portion of the upper, bodyside surface of the topsheet 26 that overlays the upper, bodyside surface of the absorbent.

The topsheet 26 may be maintained in secured relation with the backsheet 28 and/or absorbent structure 30 by bonding all or a portion of the adjacent surfaces to one another. A variety of bonding techniques known to one of skill in the art may be utilized to achieve any such secured relation. Examples of such techniques include, but are not limited to, the application of adhesives in a variety of patterns between the two adjoining surfaces, entangling at least portions of the adjacent surface of the absorbent with portions of the adjacent surface of the topsheet, or fusing at least portions of the adjacent surface of the topsheet to portions of the adjacent surface of the absorbent.

The topsheet 26 extends over the upper, bodyside surface of the article, and typically extends over any employed absorbent structure to provide a bodyside liner. The topsheet can optionally extend around the article to partially or entirely, surround or enclose the absorbent structure. Alternatively, the topsheet 26 and the backsheet 28 can have peripheral margins which extend outwardly beyond the terminal, peripheral edges of the absorbent structure 30, and the extending margins can be joined or otherwise operatively connected together to partially or entirely, surround or enclose the absorbent structure.

The baffle or backsheet 28 may include a layer constructed of any operative material, and may or may not be configured to be liquid-permeable. In a particular configuration, the backsheet 28 may be configured to provide an operatively liquid-impermeable layer. The backsheet may, for example, include a polymeric film, a woven fabric, a nonwoven fabric or the like, as well as combinations or composites thereof. For example, the backsheet may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the backsheet 28 can operatively permit a sufficient passage of air and moisture vapor out of the article, particularly out of an absorbent (e.g. the storage or absorbent structure 30) while blocking the passage of bodily liquids. An example of a suitable backsheet material can include a breathable, microporous film, such as a HANJIN Breathable backsheet available from Hanjin Printing, Hanjin P&C Company Limited, a business having offices located in Sahvon-Ii.Jungan-mvu.Kongiu-City, Chung cheong nam-do, Republic of South Korea. The backsheet material is a breathable film, which is white in color, dimple embossed, and contains: 47.78% calcium carbonate, 2.22% TiO₂, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed cell polyolefin foam. For example, a closed cell polyethylene foam may be employed. Still another example of a backsheet material would be a material that is similar to a polyethylene film which is used on commercially sold KOTEX brand pantiliners. Suitable film materials have been obtainable from Pliant Corporation, a business having offices located in Schaumburg, Ill., USA.

The structure of any employed absorbent body 30 can be operatively configured to provide a desired level of absorbency or storage capacity. More particularly, the absorbent body can be configured to hold a liquid, such as urine, menses, other complex liquids or the like, as well as combinations thereof. As representatively shown, the absorbent body can include a matrix of absorbent fibers and/or absorbent particulate material, and the absorbent fiber can include natural and/or synthetic fiber.

Any employed absorbent structure 30 may also include superabsorbent material. Superabsorbent materials suitable for use in the present invention are known to those skilled in the art, and may be in any operative form, such layers, fibers, particles or the like, as well as combinations thereof. Generally stated, the water-swellable, generally water-insoluble, hydrogel-forming polymeric absorbent material (superabsorbent) is capable of absorbing at least about 10, desirably about 20, and possibly about 100 times or more its weight in water. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company, Hoechst Celanese Corporation, Allied Colloid Inc., and Stockhausen, Inc.

The absorbent body 30 can be substantially unitary with a non-uniform structure or a generally uniform structure. Alternatively, the absorbent body may include a composite structure having a selected plurality of strata or layers. For example, the absorbent body structure may include an intake layer, a distribution layer, a transfer layer, a transfer-delay layer, a shaping layer, a retention layer or the like, as well as combinations thereof. The various strata and/or layers may be stacked, distributed or otherwise arranged in any operative sequence or configuration.

Additionally, the absorbent article can include any desired pattern or array of embossments. In particular aspects, the embossments may be formed on the bodyside surface of the article. Desired arrangements can include an absorbent body structure that has embossment regions formed on at least its bodyside surface. Similarly, the other employed components of the article can also include corresponding embossed regions.

In desired configurations, the absorbent body 30 can provide an overall, total absorbent capacity which is at least a minimum of about 1 or 2 grams of menses simulant. The overall absorbent capacity can alternatively be at least about 10 grams or at least about 20 grams of menses simulant, and can optionally be at least about 30 grams of menses simulant to provide improved performance. In other aspects, the overall absorbent capacity can be up to a maximum of about 120 grams of menses simulant, or more. The overall absorbent capacity can alternatively be up to about 100 grams of menses simulant, and can optionally be up to about 90 grams of menses simulant to provide improved effectiveness.

The article 20 can include a system of side “wings” or wing-panel portions 42 which are positioned along both lateral side regions 64 of the article. The wing-panels can be separately provided members that are subsequently attached or otherwise operatively connected to the intermediate portion of the article 20. The separately provided wing-panel member can be operatively attached to at least one of the backsheet 28 and/or topsheet 26. In an alternative arrangement, each wing-panel 42 can be provided by an integrally-formed side portion of the article 20. The wing-panels may be integrally formed from another component of the article, such as the topsheet and/or the backsheet, and joined or otherwise operatively connected to appointed sections of the article side regions 64 along the intermediate portion of the article.

In a particular configuration, each wing-panel 42 can include an integrally-formed side portion of the backsheet 28 which extends laterally past a corresponding, terminal side edge of the absorbent body 30. In another arrangement, each wing-panel 42 can include an integrally-formed side portion of the topsheet 26 which extends laterally past a corresponding, terminal side edge of the absorbent body. Still a further arrangement can have a configuration wherein each wing-panel 42 includes a panel composite. The panel composite can, for example, include an integrally-formed side portion of the backsheet 28 which extends laterally past a corresponding, terminal side edge of the absorbent body, and an integrally-formed cooperating, side portion of the topsheet 26 which extends laterally past the corresponding, terminal side edge of the absorbent body. The cooperating, side portion of the topsheet 26 is positioned in facing relation with the integrally-formed side portion of the backsheet 28.

In the cooperating pair of wing-panels, the first wing-panel 42 can be attached or otherwise operatively connected to a first side edge region 64 in the intermediate portion 76 of the article 20, and the second wing-panel 42a can be attached to a second side edge region 64a in the intermediate portion of the article. Each wing-panel 42 (42, 42a) can be configured to operatively wrap about its corresponding side edge of a wearer's undergarment.

The wing-panels can have an appointed storage position in which the wing-panels 42 are directed generally inwardly toward the longitudinally-extending centerline 52. The wing-panel that is connected to extend from one side margin may have sufficient cross-directional length to extend and continue past the centerline 52 to approach the laterally opposite side margin of the article. The storage position of the wing-panels can ordinarily represent an arrangement observed when article is first removed from its wrapper or other packaging. Prior to placing the article into a bodyside of an undergarment prior to use, the wing-panels 42 can be selectively arranged to extend laterally from the side regions 64 of the article intermediate portion. After placing the article in the undergarment, the wing-panels 42 can be operatively wrapped and secured in an in-use position around the side edges of the undergarment crotch portion to help hold the article in place. Typically, the wing-panels are configured to secure the article to a crotch portion of the wearer's undergarment.

The wing-panel portions 42 can have any operative construction, and can include a layer of any operative material. Additionally, each wing-panel can comprise a laminate or other composite material. For example, the wing-panels may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

The wing-panel materials can be substantially non-stretchable or may be stretchable. The wing-panel material may also be capable of providing a selected amount of elastomeric stretch and retraction. In particular configurations, the wing-panel material can exhibit a maximum stretch elongation value of up to about 300%, or more. In other configurations, the wing-panel material can exhibit a minimum stretch elongation value of 50%. In other features, the wing-panel material can have a basis weight which is within the range of about 0.5-3 ounces per square yard (about 17-102 g/m²). By employing such wing-panel materials, the wing-panel can help provide desired fit characteristics, and can help provide improved leak protection.

Each wing-panel 42 can include a panel-fastener component 44 which is operatively connected to a major facing surface of the associated wing-panel. Such major surface is typically appointed to engage and become operatively attached to an outward-facing surface of the wearer's undergarment. The panel-fastener 44 can include any operative fastener component, such as a component of an interengaging mechanical faster, an adhesive fastener, a cohesive fastener, a magnetic fastener, an electromechanical fastener or the like, as well as combinations thereof. The mechanical fastener component may, for example, include a suitable hook component, such as a J-hook, mushroom-head hook, flat-top nail-head hook, a palm-tree hook, a multiple-J hook or the like, as well as combinations thereof. Optionally, the mechanical fastener component may, for example, include a suitable loop component. The loop component may, for example, include a knit fabric, a woven fabric, a nonwoven fabric or the like, as well as combinations thereof.

Each wing-panel 42 can include a panel base section and a panel free-end region. Each panel base section can be operatively connected to its corresponding side edge region 64 of the article 20, and each panel free-end region can extend from its corresponding panel base section. The free-end region can be further configured to operatively wrap around an appointed region of the wearer's undergarment. For example, the free-end regions of the wing-panels can be wrapped about a crotch region of the wearer's undergarment.

With reference to FIGS. 4, 9 and 11, a selected configuration of a garment-fastener or other garment-attachment mechanism 38 may be operatively distributed and connected onto the garment-side surface of each article 20 to help secure that article to the wearer's undergarment. The garment-fastener may be arranged any operative pattern, such as one or more strip regions that are distributed onto the garment-side of the article. As representatively shown, each article 20 can include a garment-fastener 38 which is operatively attached to a garment-side surface of its corresponding backsheet 28. In a desired arrangement, the garment-fastener 38 can be substantially fixedly attached to the garment-side surface of its corresponding backsheet 28 with a factory-bond, and can be configured to releasably secure that article to the wearer's undergarment. Additionally, each article 20 can further include a release layer 40 which covers the corresponding garment-fastener 38 of that article. In a desired configuration, at least a portion of the release layer of a primary article 74 can be releasably connected to at least a portion of the release layer of a serially-adjacent, sequential-article 78.

The garment-fastener can include any operative fastener mechanism, such as a component of an interengaging mechanical fastener, an adhesive fastener, a cohesive fastener, a magnetic fastener, and electromechanical fastener or the like, as well as combinations thereof. For example, the garment-fastener can be provided by the representatively shown adhesive 38a, which may be arranged any operative pattern, such as the representatively shown strip regions that are distributed onto the garment-side of the article. In particular arrangements, an operative garment adhesive can be distributed over the garment-side of the backsheet, and one or more layers or sheets of release material 40 can be removably placed over the garment adhesive during storage prior to use. In desired arrangements, at least a significant portion of the garment-fastener 38 can be generally aligned generally parallel to the longitudinal centerline 52 of the article.

With reference to FIGS. 1 and 5, each article 20 can provide a first major face-surface 46, and a second major face-surface 48 positioned opposite the first surface 46. Each article can also include at least a first transverse fold 66, which provides the article with a first section 56 and a second section 58. The first transverse fold can be configured to place a first section 56 of a first major face-surface 46 of that article in a position that is substantially adjacent a second section 58 of the first surface 46 of that article to thereby provide a folded article. In a desired arrangement, for example, the first transverse fold 66 can be configured to place a first region or section 56 of a bodyside surface of the topsheet 26 of its corresponding article substantially adjacent a second region or section 58 of the bodyside surface of the topsheet 26 of that article to provide the folded article. As representatively shown (e.g. FIGS. 1 and 8), each sequence-attachment mechanism 34 can releasably connect a second section 58 of a primary-article 74 to a first section 56 of a serially-adjacent, sequential-article 78.

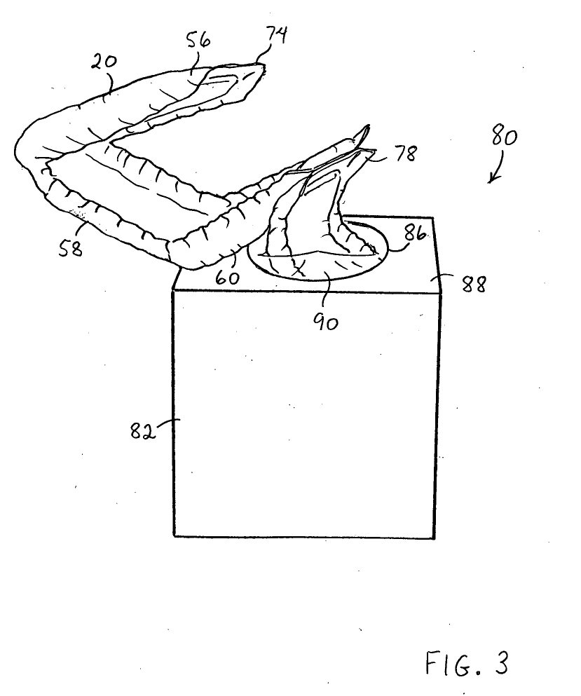

With reference to FIGS. 1 and 3, each article 20 can include a first transverse fold 66 and at least a second transverse fold 68. As a result, each article 20 can further include at least a third section 60. In a particular feature, the first transverse fold 66 can overlay the first section 56 of the article 20 in a position that is substantially adjacent the second section 58 of that article. In another aspect, the second transverse fold 68 can place the third second 60 of that article in a position that is over at least a portion of the second section 58 of that article. Additionally, each sequence-attachment mechanism can releasably connect a third section 60 of a primary-article 74 to a first section 56 of a serially-adjacent, sequential-article 78.

Each releasable, sequence-attachment mechanism 34 can releasably join a base, primary-article 74 to a serially-adjacent, sequential-article 78. In the various configurations of the invention, the releasable, sequence-attachment mechanism that is associated with the first, primary article 74 can releasably join a garment-side or bodyside surface of the primary-article 74 to a garment-side or bodyside surface of a serially-adjacent, sequential-article 78. The connection provided by the sequence-attachment mechanism is sufficiently strong to allow an operative entrainment of the articles during their manufacture and packaging, and during their dispensing from the employed package 82. The connection is also sufficiently weak to allow a ready separation between individual articles or between selected groups of articles, as desired by the user. The sequence-attachment mechanism 34 can be provided by any operative attachment mechanism. For example, the sequence-attachment mechanism can include an adhesive, a cohesive, an operative component of an interengaging mechanical fastener, or the like, as well as combinations thereof.

As representatively shown, each releasable, sequence-attachment mechanism 34 can be operatively connected to the backsheet 28 of its corresponding article 20. It should be readily appreciated that the sequence-attachment mechanism can be directly or indirectly attached to the backsheet 28 of its corresponding article 20. In a particular aspect, at least a portion of the backsheet 28 of a primary article 74 can be releasably connected to at least a portion of the backsheet 28 of a serially-adjacent, sequential-article 78. In another aspect, each releasable, sequence-attachment mechanism 34 can be operatively connected to the topsheet 26 of its corresponding article 20 with a direct or indirect attachment. For example, at least a portion of the topsheet 26 of the primary article 74 can be releasably connected to at least a portion of the topsheet 26 of the serially-adjacent, sequential-article 78.

In the various configurations of the invention, the releasable, sequence-attachment mechanism 34 of each article 20 can include a separately provided sequencing-component that releasably interconnects between the base, primary-article 74 and its associated, serially-adjacent, sequential-article 78. For example, the sequence-attachment mechanism can include a separately provided adhesive, a separately provided cohesive, a separately provided component of an interengaging mechanical fastener, or the like, as well as combinations thereof.

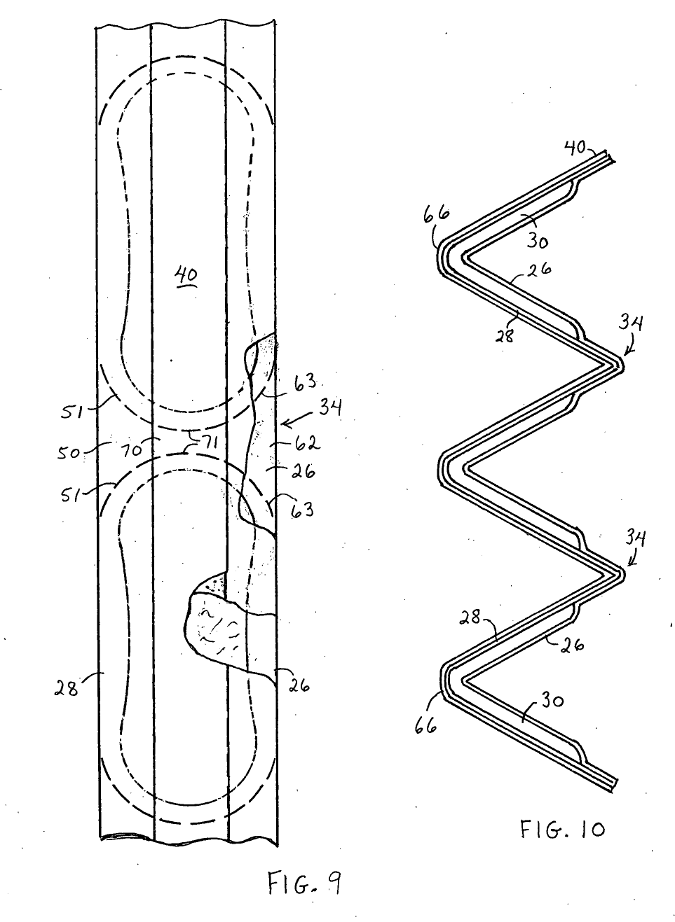

A particular aspect of the invention can have a construction in which the sequence-attachment mechanism 34 is provided by a selected modification or other adjusted configuration of an existing, previously present component of the article 20. A particular arrangement can, for example, include a configuration wherein at least a portion of the backsheet 28 of a primary-article 74 is connected to at least a portion of the backsheet of a serially-adjacent, sequential-article 78 along a backsheet joinder region 50. The backsheet joinder region can include an operative, frangible line of weakness 51 to thereby provide at least a portion of a releasable, sequence-attachment mechanism between the primary-article 74 and the sequential-article 78, as representatively shown in FIGS. 9 and 10.

Another aspect can include a configuration wherein at least a portion of the topsheet 26 of a primary-article 74 is connected to at least a portion of the topsheet 26 of serially-adjacent, sequential-article 78 along a topsheet joinder region 62. The topsheet joinder region can include an operatively frangible line of weakness 63 to thereby provide at least a portion of a releasably, sequence-attachment mechanism 34 between the primary article 74 and the sequential-article 78 (e.g. FIGS. 9 and 10).

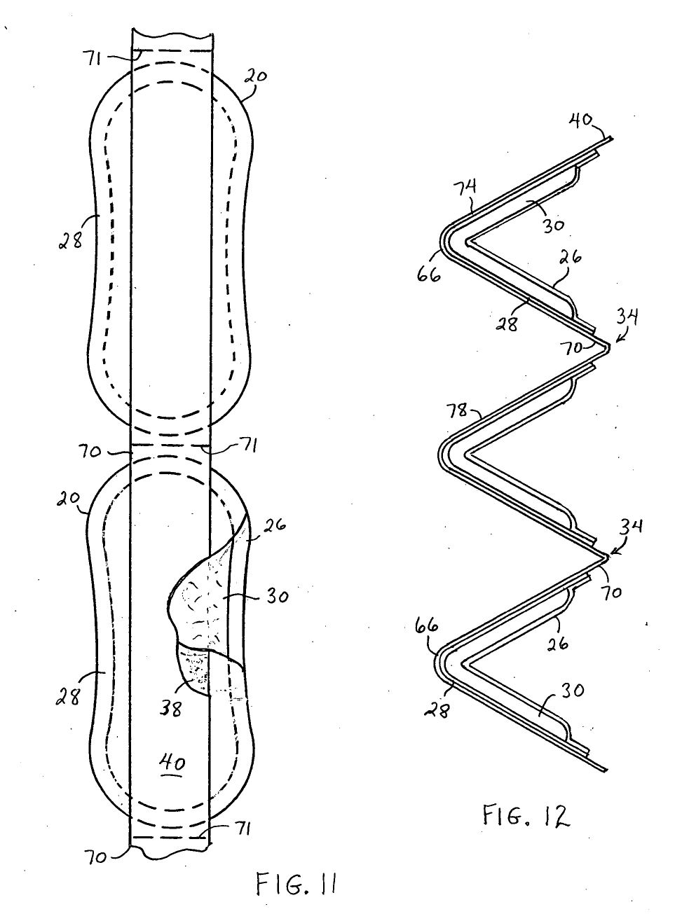

In a further aspect, each article 20 can further include a garment-fastener 38 and a release layer 40 which operatively covers the garment-fastener 38 of that particular article 20. As representatively shown, the release layer 40 can, for example, be provided by a separately provided peel strip of material. At least a portion of the release layer 40 of a primary article 74 can be joined or otherwise operatively connected to at least a portion of the release layer of a serially-adjacent, sequential-article 78 along a strip joinder region 71. The strip joinder region can include an operatively frangible line of weakness 71 to thereby provide at least a portion of a releasable, sequence-attachment mechanism 34 between the primary-article 74 and the sequential-article 78, as representatively shown in FIGS. 11 and 12.

In particular arrangements, the primary-article 74 can be connected to at least a portion of any desired combination of the backsheet 28, topsheet 26 and/or release layer 40 of the serially-adjacent, sequential-article 78 along the desired combination of the corresponding backsheet joinder region, topsheet joinder region and/or strip joinder region. Each of the backsheet, topsheet and/or strip joinder regions can include an corresponding, frangible line of weakness to thereby provide at least a portion of the releasable, sequence-attachment mechanism between the selected primary-article 74 and its corresponding sequential-article 78.

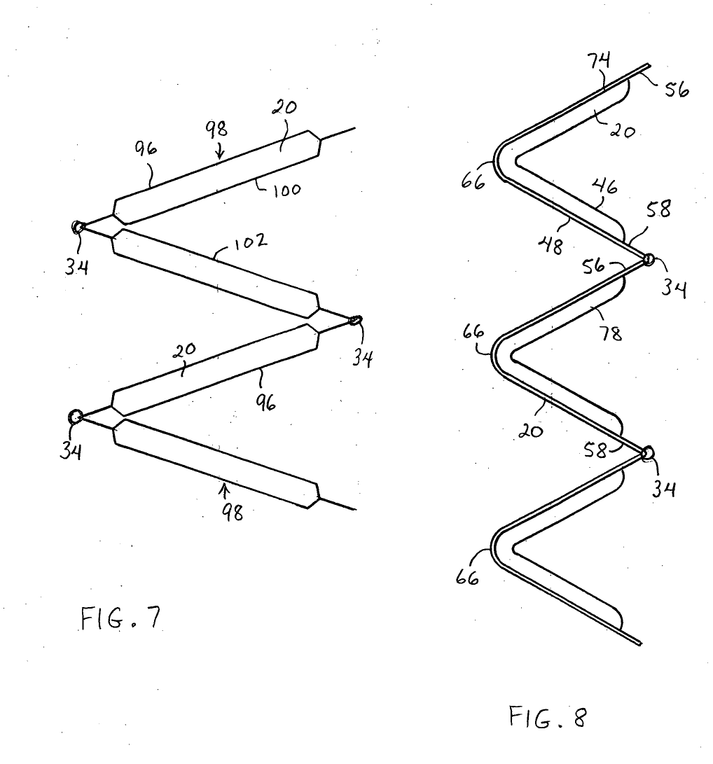

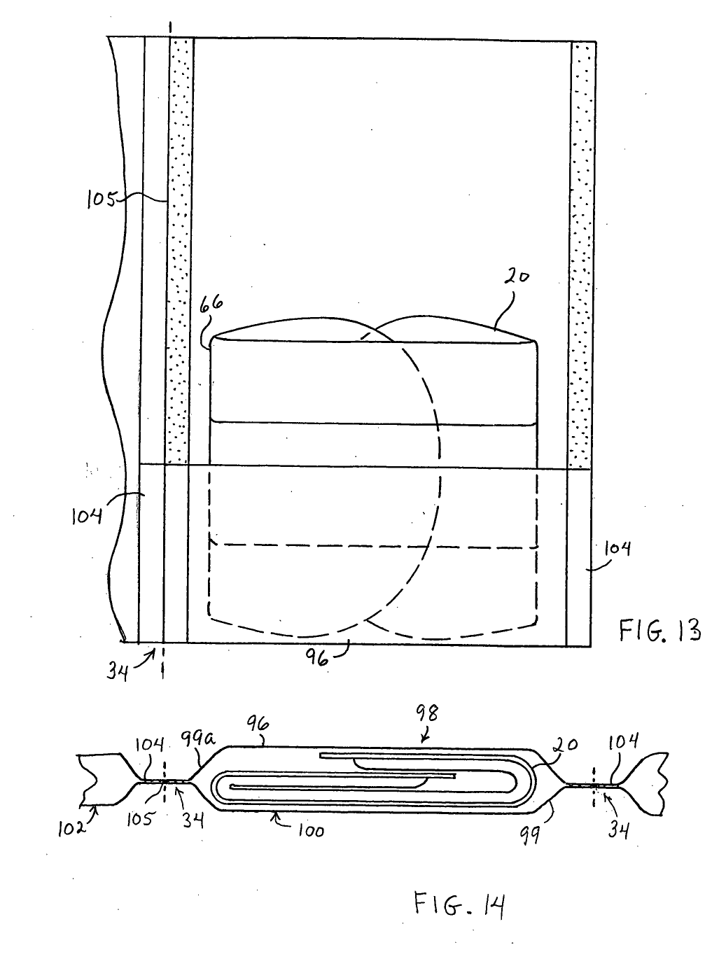

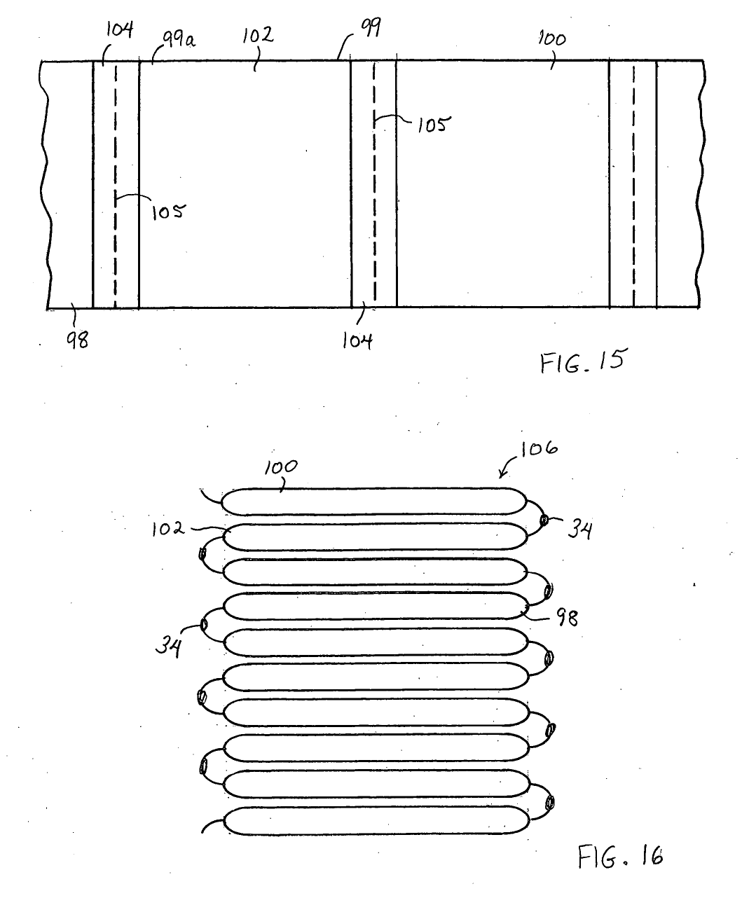

With reference to FIGS. 7, 13, 14 and 15, each article 20 may further include a wrapper 96 which can operatively enclose that particular article. Additionally, the releasable, sequence-attachment mechanism 34 of that particular article can be operatively connected to at least a portion of the wrapper 96 of that article. As representatively shown, each wrapper 96 can operatively provide a wrapped enclosure about its corresponding article, and each article 20 may include at least a first transverse fold 66 when positioned in its corresponding wrapped enclosure.

As representatively shown, each wrapped enclosure 98 can include a first end portion 99 and a longitudinally opposed, second end portion 99a. Each sequence-attachment mechanism 34 can releasably connect an end portion (99, 99a) of a base or primary wrapped enclosure 100 to an end portion (99, 99a) of a serially-adjacent, sequential wrapped enclosure 102. In a particular aspect, the wrapper and wrapped enclosure that is associated with a primary-article 74 can be connected to the wrapper and wrapped enclosure that is associated with a serially-adjacent, sequential-article. The connection can desirably extend along a wrap-joinder region 104, and the wrap-joinder region can include an operative, frangible line of weakness 105 to thereby provide at least a portion of a releasable, sequence-attachment mechanism 34 between the primary-article 74 and its corresponding sequential-article 78.

The selected frangible line of weakness (51, 63, 105) can be provided by any operative technique or construction. The line of weakness can, for example, be provided by a thinning of the material of the described component, a scoring of the material, a perforating of the material, a physical weakening of the material, a chemical weakening of the material, a thermal weakening of the material or the like, as well as combinations thereof. The line of weakness may have a configuration that is discontinuous or substantially continuous, and may have an arrangement that is nonlinear, curvilinear or substantially linear, as well as combinations thereof. Additionally, the line of weakness can include elements that are arranged in any operative distribution. For example, elements of the line of weakness may be distributed in an irregular or substantially regular array, as desired.

In the various configurations of the invention, any operative package 82 may be employed. The package 82 can, for example, include a bag, and in desired arrangements, the bag can be operatively flexible and readily collapsible. In particular arrangements, the bag can be collapsible under its own weight. The package 82 can optionally be provided by a box, carton or the like, and in particular arrangements the package can be semi-rigid or substantially rigid, as desired.

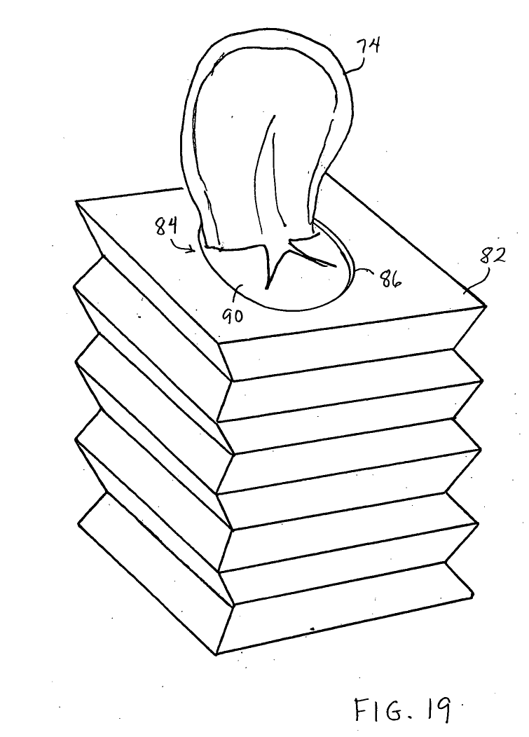

With reference to FIGS. 1, 2 and 3, the exit mechanism 84 can include a package face-panel 88, which may be a top panel, a side panel or a bottom panel, as desired. The selected face-panel can include a package opening 86 having a boundary edge, and at least a major portion of the boundary edge can be curvilinear. In another aspect, the exit mechanism 84 can include an operatively flexible panel-member 90 which provides an exit slot 92 therethrough. In a particular aspect, the flexible panel-member 90 can be configured to span across the package opening 86. In other aspects, the panel-member 90 can include an exit slot 92, and the exit slot can have a slot opening which is smaller than the package opening 86. The boundary edge of the package opening 86 can have any operative shape. The shape may be regular, irregular, rectilinear, curvilinear, geometric, non-geometric and the like, as well as combinations thereof. Additionally, selected portions of the boundary edge may be concave or convex. As representatively shown (e.g. FIGS. 2 and 18), for example, the boundary edge of the package opening 86 can be generally oval-shaped. Optionally, one or more predetermined sections of the boundary edge may be concave with one or more cooperating sections of the corresponding face-panel 88 protruding into the package opening 86. The protruding section of the face-panel can be configured to operatively contact or otherwise engage a trailing sequential article 78 to help separate the sequential article from its relatively preceding primary article 74. For example, a selection portion, such as a distal edge, of the protruding section of the face-panel may be serrated or otherwise treated or configured to provide a friction-enhancing mechanism. The provided engagement can also help to the keep the sequential article from falling back into the package 82 and help present and maintain that article in a position ready for subsequent grasping by the user.

In desired arrangements, a plurality of at least 2 individual articles 20 can be contained in the package 82. Each package can alternatively contain at least about 2 or 3 individual articles, and can optionally contain at least about 5 individual articles. In other arrangements, each package can alternatively contain at least about 10 individual articles, and may optionally contain at least about 15 individual articles. In still further arrangements, each package can contain at least about 25 individual articles, and may optionally contain at least about 45 individual articles. Additionally, each package can contain a maximum of up to about 100 or more of individual articles.

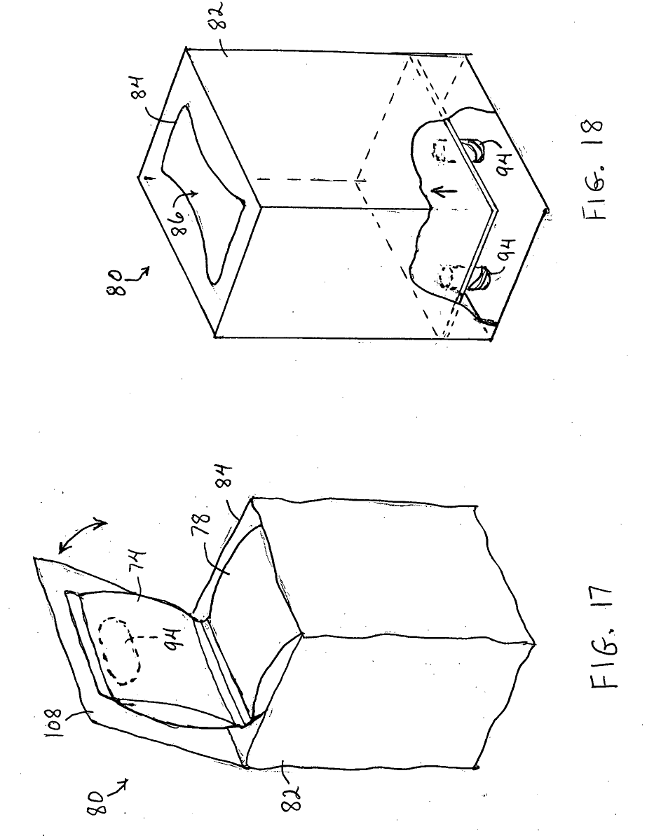

With reference to FIGS. 17 and 18, the dispensing system 80 can further include a position-advancement mechanism 94 which can provide an operative proximity between a next-unused article and the exit mechanism 84 of the package 82. More particularly, the position-advancement mechanism can be configured to help move and hold the next article in an operative proximity and closeness to the exit opening 86 of the package. Having the articles close to the opening of the package can help save time by conveniently presenting articles near at hand for use. Multi-functional lids, spring devices, raising levers, pull strips and/or other components can aid in moving the articles 20 toward the opening 86 of the package 82 for easy access.

The package 82 can, for example, include a flap or lid 108, and the package flap can include an operatively connected traction device which provides an advancement mechanism that can suitably engage and pull up a single article at a time for convenient dispensing (e.g. FIG. 17). The traction device may be provided by a hook material, a microhook material, a low-tack, readily releasable adhesive, a pin, a tack or the like, as well as combinations thereof. The dispensing system can, for example, include an attachment zone on the flap, and the selected traction device can be located on the attachment zone. The attachment zone can be configured to operatively engage and pull up one article at a time for dispensing when the flap is moved from a closed position to an open position.

In an alternative aspect, an operative forcing mechanism can be incorporated in the package 82 to help move the articles 20 toward the package opening 86 (e.g. FIG. 18). The position-advancement mechanism can include a forcing mechanism, such as a spring device or the like, As representatively shown, the forcing mechanism can be incorporated at the bottom of the package 82. The forcing mechanism can optionally be positioned at any other operative location in the employed package, such as at the top wall or side walls of the package. The position-advancement mechanism can be appropriately configured to thereby provide a force which operatively urges the fresh, unused articles within the package 82 toward the exit mechanism 84. The position-advancement mechanism can provide a force that is sufficiently high to operatively move the weight of the contained articles toward the exit mechanism of the dispensing system, but is sufficiently low to avoid an undesired ejection of articles from the package 82. In optional arrangements, the position-advancement mechanism can, for example, be provided by one or more of the following devices: a mechanical spring, a pneumatic spring, a magnetic actuator, an electromechanical actuator, an elastomeric actuator, a pneumatic actuator or the like, as well as combinations thereof.

Desired arrangements can have an exit mechanism 84 which is configured to provide a operative, frictional drag on the articles 20 as the articles are moved through the exit mechanism (e.g. FIGS. 2 and 17). The selected level of frictional drag can help the user generate the desired separation of one or more individual articles from the remaining articles intended to be left in the package 82. Additionally, the frictional drag can help retain the articles in the package even when the articles are subjected to the urging of any employed forcing mechanism. The generation of the desired level of frictional drag can be obtained by any operative technique or construction, and such techniques and constructions are well known in the art. For example, the size and shape of the package opening 86, the material of the panel-member 90 and the size and shape of the slot 92 can be appropriately adjusted to provide the desired level of frictional drag.

Optional package designs or package materials and components can be employed to improve the ease of dispensing. For example, the dispensing system can be configured to either move the bottom of the package closer to the top of the package, or to provide package materials or package sides which allow the top of the package to collapse down on itself. To help the package collapse, the top of the package can be configured to have sufficient weight and the sides of the package can be configured to be sufficiently non-rigid or flexible to produce the desired collapse of the package. For example, the sides of the package can be configured to be contractible or shrinkable. Optionally, the package sides can be configured to accordion-fold down to a small height.

With reference to FIG. 19, the package 82 can include an operative set of cooperating side wall members, and the wall members can be configured to help provide at least a portion of the position-advancement mechanism. For example, the side wall members can include an operative array of accordion folds, which can be selectively extended and collapsed. As a result, the size and volume of the package 82 can be gradually reduced as individual articles 20 are removed from the package and the number of unused articles within the package is reduced.

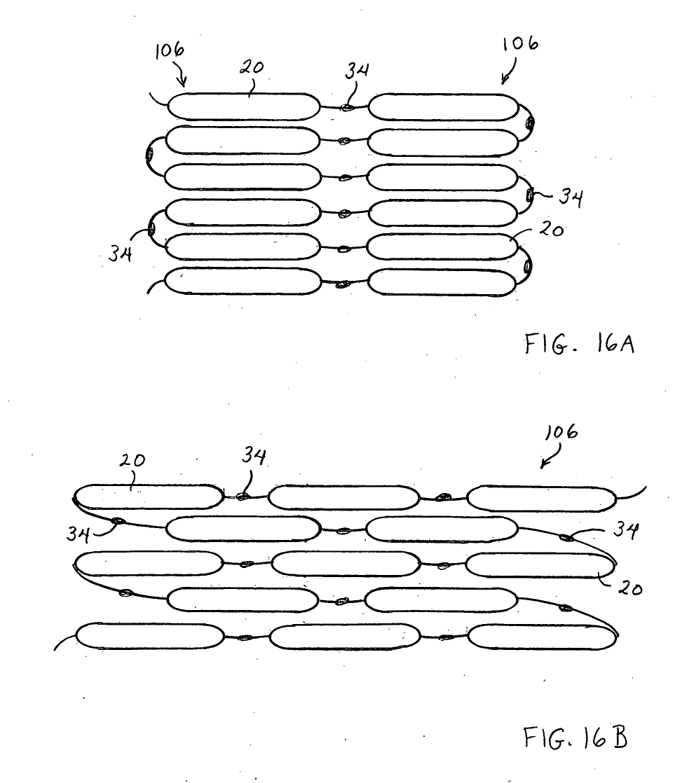

Any operative zig-zag arrangement of the articles 20 can be employed, and the individual articles 20 can, for example, be arranged to provide a desired array of article stacks 106 within an individual package 82. An individual package 82 can contain only a single stack 106 of articles (e.g. FIGS. 1 and 16). Alternatively, the zig-zag arrangement of the articles 20 can be configured to provide an array of two or more article stacks 106 within an individual package 82 (e.g. FIGS. 16A and 16B).

In the construction of the various configurations of the dispensing system 80 and/or article 20, it should be readily appreciated that the various elements and components may be assembled and held together with any operative securement mechanism or system. For example, the desired attachments or securements can include adhesive bonds, cohesive bonds, thermal bonds, ultrasonic bonds, pins, snaps, staples, rivets, stitches, welds, zippers, or the like, as well as combinations thereof.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. An article dispensing system comprising a package; and a plurality of personal care articles contained in said package; wherein
each article includes a topsheet, and a backsheet operatively connected with the topsheet;
each article includes a releasable, sequence-attachment mechanism operatively connected to that article;
said articles have been connected to provide a sequencing of the articles in an operative zig-zag arrangement; and
said package includes a exit mechanism which provides a package opening through which the articles can be removed from said package.

2. A dispensing system as recited in claim 1, wherein each article includes at least one transverse fold which places a first section of a first surface of said each article substantially adjacent a second section of said first surface of a said each article, thereby providing a folded article.

3. A dispensing system as recited in claim 1, wherein each releasable, sequence-attachment mechanism is operatively connected to the backsheet of its corresponding article.

4. A dispensing system as recited in claim 1 wherein at least a portion of a backsheet of a primary-article is releasably connected to at least a portion of a backsheet of a serially-adjacent, sequential-article.

5. A dispensing system as recited in claim 1 wherein at least a portion of a topsheet of a primary-article is releasably connected to at least a portion of a topsheet of a serially-adjacent, sequential-article.

6. A dispensing system as recited in claim 1, wherein each article includes at least one transverse fold which places a first section of a bodyside surface of the topsheet of that article substantially adjacent a second section of said bodyside surface of the topsheet of that article, thereby providing a folded article.

7. A dispensing system as recited in claim 1, wherein the releasable, sequence-attachment mechanism of a primary-article releasably attaches a garment-side or bodyside surface of the primary-article to a garment-side or bodyside surface of a serially-adjacent, sequential-article.

8. A dispensing system as recited in claim 1, wherein each article includes a first transverse fold and at least a second transverse fold.

9. A dispensing system as recited in claim 8, wherein
each article further includes at first section, a second section, at least a third section, and has a second surface positioned opposite said first surface;
the first transverse fold overlays the first section of said each article in a position that is substantially adjacent the second section of said each article;
the second transverse fold places the third section of said each article in a position that is over at least a portion of the second section of said each article;
each sequence-attachment mechanism releasably connects a third section of a primary-article to a first section of a serially-adjacent, sequential-article.

10. A dispensing system as recited in claim 8, wherein the sequence-attachment mechanism releasably joins a garment-side or bodyside surface of a primary-article to a garment-side or bodyside surface of a serially-adjacent, sequential-article.

11. A dispensing system as recited in claim 1, wherein the releasable, sequence-attachment mechanism of said each article includes a separately provided sequencing-component that releasably interconnects between a primary-article and a serially-adjacent, sequential-article.

12. A dispensing system as recited in claim 1, wherein the releasable, sequence-attachment mechanism of said each article includes an adhesive.

13. A dispensing system as recited in claim 1, wherein
at least a portion of a backsheet of a primary-article is connected to at least a portion of a backsheet of a serially-adjacent, sequential-article along a backsheet joinder region;
the backsheet joinder region includes an operative, frangible line of weakness to thereby provide at least a portion of a releasable, sequence-attachment mechanism between the primary-article and the sequential-article.

14. A dispensing system as recited in claim 1, wherein
at least a portion of a topsheet of a primary-article is connected to at least a portion of a topsheet of a serially-adjacent, sequential-article along a topsheet joinder region;
the topsheet joinder region includes an operatively frangible line of weakness to thereby provide at least a portion of a releasable, sequence-attachment mechanism between the primary-article and the sequential-article.

15. A dispensing system as recited in claim 1, wherein
each article further includes
a garment-fastener which is substantially fixedly attached to a garment-side surface of its corresponding backsheet, and configured to operatively secure that article to a wearer's undergarment, and
a release layer which operatively covers the garment-fastener of that article;
at least a portion of the release layer of a primary-article is connected to at least a portion of the release layer of a serially-adjacent, sequential-article along a strip joinder region; and
the strip joinder region includes an operatively frangible line of weakness to thereby provide at least a portion of a releasable, sequence-attachment mechanism between the primary-article and the sequential-article.

16. A dispensing system as recited in claim 1, wherein
each article further includes a wrapper which operatively provides a wrapped enclosure about that article, and
each article includes at least one transverse fold when positioned in its corresponding wrapped enclosure.

17. A dispensing system as recited in claim 1, wherein
each article further includes a wrapper which operatively provides a wrapped enclosure about that article;
each wrapped enclosure has a first end portion and a longitudinally opposed second end portion;
each sequence-attachment mechanism releasably connects an end portion of a primary wrapped enclosure to an end portion of a serially-adjacent wrapped enclosure.

18. A dispensing system as recited in claim 1, wherein
each article further includes a wrapper which operatively provides a wrapped enclosure about that article;
each wrapped enclosure has a first end portion and a longitudinally opposed second end portion;
a wrapped enclosure of a primary-article is connected to a wrapped enclosure of a serially-adjacent sequential article along a wrap-joinder region;
the wrap-joinder region includes an operatively frangible line of weakness to thereby provide at least a portion of a releasable, sequence-attachment mechanism between the primary-article and the sequential-article.

19. A dispensing system as recited in claim 1, wherein the exit mechanism includes:
a package face-panel which includes a package opening having a boundary edge, at least a major portion of which is curvilinear; and
an operatively flexible panel-member which spans across the package opening and provides an exit slot, the exit slot having a slot opening which is smaller than said package opening

20. A dispensing system as recited in claim 1, further including a position-advancement mechanism which provides an operative proximity between a next-unused article and the exit mechanism of the package.
